(19) United States
(12) Patent Application Publication
(10) Pub. No.: US 2020/0146555 A1
(43) Pub. Date: May 14, 2020

(54) DEVICES, SYSTEMS, AND METHODS FOR CONTROLLING DISTANCE BETWEEN AN IMAGING DEVICE AND A WOUND

(71) Applicant: Atlantic Health System, Inc., Morristown, NJ (US)

(72) Inventor: Mildred Ortu Kowalski, East Hanover, NJ (US)

(21) Appl. No.: 16/403,199

(22) Filed: May 3, 2019

Related U.S. Application Data
(60) Provisional application No. 62/760,645, filed on Nov. 13, 2018.

(51) Int. Cl. A61B 5/00

(52) U.S. Cl. CPC A61B 5/0077; A61B 5/0064; A61B 5/6898; A61B 5/445; A61B 5/4216; A61B 5/6844

(57) ABSTRACT

An apparatus for measuring a distance includes a distance measurement device, a light source, and a photographic device holder portion. The photographic device holder portion includes a first region having a first surface at a first level, and a second region having a second surface at a second level different from the first level. The first region and second region are adapted to hold a photographic device, such as a cellular telephone, in place on the apparatus. The apparatus may include a layer of a selected material disposed at an edge of the first region, wherein the material is adapted to hold the photographic device in place. The apparatus may include a main body section and an extendable section adapted to slide in and out relative to the body section in order to accommodate different sizes of cellular telephones.

### TECHNICAL FIELD

This specification relates generally to the medical field, and more particularly to devices, systems, and methods for controlling distance between an imaging device and a wound and consistency of the resulting image.

### BACKGROUND

In hospitals and other medical facilities where wounds are treated, it is often desirable to capture multiple images of a wound at selected times to document how the wound appears at different stages of treatment. For example, an initial photograph of a pressure injury or skin ulcer may be captured; subsequently, during treatment, additional photographs of the wound may be captured at various times to document how the wound is healing. Photographs provide an objective view of a wound, and are therefore preferable in comparison to reliance on narrative text documentation, which may be subjective and therefore relatively unreliable.

To observe how a wound changes over time, multiple photographs are often taken at different times. To ensure consistency and to develop a record that is accurate and meaningful, it is important to establish as much consistency as possible in the manner used to photograph the wound or injury. In particular, photographs taken at different times should be taken from the same distance and under similar lighting conditions.

Existing methods of photographing wounds do not control for these parameters and therefore fail to guarantee consistency or reliability. In many hospitals and medical facilities, each time a photograph of a wound is desired, a nurse or other practitioner simply holds a camera by hand and takes a picture of the wound, choosing a distance that seems at the time to provide a clear view of the wound. No attempt is made to ensure that the distance between the camera and the wound is consistent for each photograph of a particular wound. Similarly, no attempt is made to ensure that each photograph of a particular wound is made under similar lighting conditions. As a result, photographs taken of a particular wound are typically taken at different distances, and under different light conditions, and as a result may prevent meaningful comparisons.

There is a need for devices and methods that standardize the distance between a wound and the camera when multiple photographs of the wound are taken. There is a further need for devices and methods that standardize the distance between a wound and a camera when the camera is placed at a predetermined angle (e.g., 90 degrees) relative to the wound. There is also a need for devices and methods that standardize the lighting conditions used in taking photographs of a particular wound at different times.

### SUMMARY

Advantageously, apparatus and methods for taking multiple photographs of a wound at different times, from the same distance and same angle, and under similar lighting conditions, can provide more meaningful comparisons and therefore more valuable information than photographs taken using existing methods (which do not guarantee consistency in distance or lighting conditions).

In accordance with an embodiment, an apparatus for measuring a distance includes a photographic device holder portion, a distance measurement device, and a light source.

In one embodiment, the photographic device holder portion includes a first region having a first surface at a first level, and a second region having a second surface at a second level different from the first level. The first region and second region are adapted to hold a photographic device in place on the apparatus.

In another embodiment, the photographic device holder portion is adapted to hold a cellular telephone.

In another embodiment, the apparatus also includes a wall disposed at a boundary of the second region and between the first region and the second region, and a layer of an elastomeric material is disposed between the wall and the first region.

In another embodiment, the distance measurement device comprises a laser measurement device. In another embodiment, the distance measurement device includes an infrared distance measurement device.

In another embodiment, the light source includes one of an incandescent light source, a fluorescent light source, and a light emitting diode (LED) device.

In another embodiment, the apparatus includes a first section and an extendable portion adapted to retractably extend relative to the first section, thereby increasing a surface area of the first region.

In accordance with another embodiment, an apparatus for measuring a distance includes a distance measurement device, a light source, and a photographic device holder portion. The photographic device holder portion includes a first region having a first surface at a first level, and a second region having a second surface at a second level different from the first level. The first region and second region are adapted to hold a photographic device, such as a cellular telephone, in place on the apparatus.

In one embodiment, the apparatus includes a main body section and an extendable section adapted to slide in and out relative to the body section in order to accommodate different sizes of cellular telephones.

In accordance with another embodiment, the apparatus includes a stem section, a middle arm section that is joined to the stem section and is perpendicular to the stem section, and a lower arm section that is joined to the stem section, is perpendicular to the stem section, and is parallel to the middle arm section.

In another embodiment, the apparatus includes a first region having a first surface at a first level, wherein a first portion of the first region is located on the stem section, a second portion of the first region is located on the middle arm section, and a third portion of the first region is located on the lower arm section. The apparatus also includes a second region having a second surface at a second level different from the first level, the second region having a fourth portion located on the stem section and a fifth portion located on the lower arm section. The apparatus also includes a third region having a third surface at a third level different from the first level, the third region being located on the middle arm section. The first region, second region, and third region are adapted to hold a photographic device, such as a cellular telephone, in place on the apparatus.

In another embodiment, the distance measurement device includes a laser measurement device. In other embodiments, the distance measurement device includes an infrared distance measurement device.

In another embodiment, the light source includes one of an incandescent light source, a fluorescent light source, and a light emitting diode (LED) device.

In accordance with another embodiment, a method is provided. A photographic device, such as a cellular telephone, is secured in an image distance control device. The image distance control device is positioned near a wound that is to be imaged. A position of the image distance control device is adjusted until the image distance control device indicates that the distance from the image distance control device to the wound equals a predetermined distance. The photographic device (e.g., the cellular telephone) secured in the image distance control device is employed to generate an image of the wound.

These and other advantages of the present disclosure will be apparent to those of ordinary skill in the art by reference to the following Detailed Description and the accompanying drawings.

### BRIEF DESCRIPTION OF THE DRAWINGS

FIG. 1 shows functional components of an image distance control device in accordance with an embodiment;

FIG. 2 shows functional components of an image distance control device in accordance with another embodiment;

FIG. 3 shows a front view of an image distance control device in accordance with an embodiment;

FIG. 4 shows a rear view of the image distance control device of FIG. 3;

FIG. 5 shows a first side of the image distance control device of FIG. 3;

FIG. 6 shows a second side of the image distance control device of FIG. 3;

FIG. 7 shows a third side of the image distance control device of FIG. 3;

FIG. 8 shows a fourth side of the image distance control device of FIG. 3;

FIG. 9 shows a front view of a cell phone secured in the image distance control device of FIG. 3;

FIG. 10 shows a side view of a cell phone secured in the image distance control device of FIG. 3;

FIG. 11 shows another side view of a cell phone secured in the image distance control device of FIG. 3;

FIG. 12 shows an image distance control device being used to verify a distance to a wound to be imaged in accordance with an embodiment;

FIG. 13 is a flowchart of a method of using an image distance control device to determine a distance in accordance with an embodiment;

FIG. 14 is a high-level block diagram of an exemplary computer;

FIG. 15 shows a front view of an image distance control device in accordance with an embodiment;

FIG. 16 shows the image distance control device of FIG. 15 with the extendable arm section in an extended position;

FIG. 17 shows a rear view of the image distance control device of FIG. 15;

FIG. 18 shows a first side of the image distance control device of FIG. 15;

FIG. 19 shows a second side of the image distance control device of FIG. 15;

FIG. 20 shows a third side of the image distance control device of FIG. 15;

FIG. 21 shows a fourth side of the image distance control device of FIG. 15;

FIG. 22 shows a front view of the image distance control device of FIG. 15 with a cellular telephone placed therein;

FIG. 23 shows a rear view of the image distance control device of FIG. 15 with a cellular telephone placed therein;

FIG. 24 shows components of an ultrasonic rangefinder device in accordance with an embodiment;

FIG. 25 shows an ultrasonic rangefinder device in accordance with an embodiment;

FIG. 26 shows a front view of an image distance control device in accordance with an embodiment;

FIG. 27 shows a rear view of the image distance control device of FIG. 26;

FIG. 28 shows a first side of the image distance control device of FIG. 26;

FIG. 29 shows a second side of the image distance control device of FIG. 26;

FIG. 30 shows a third side of the image distance control device of FIG. 26;

FIG. 31 shows a fourth side of the image distance control device of FIG. 26;

FIG. 32 shows a front view of the image distance control device of FIG. 26 with a cellular telephone placed therein;

FIG. 33 shows a side view of the image distance control device of FIG. 26 with a cellular telephone placed therein;

FIG. 34 shows a front view of an image distance control in accordance with an embodiment;

FIG. 35 shows a rear view of the image distance control device of FIG. 34;

FIG. 36 shows a first side view of the image distance control device of FIG. 34;

FIG. 37 shows a second side view of the image distance control device of FIG. 34;

FIG. 38 shows a third side view of the image distance control device of FIG. 34;

FIG. 39 shows a fourth side view of the image distance control device of FIG. 34;

FIG. 40 shows the image distance control device of FIG. 34 with an extendable portion in an extended position;

FIG. 41 shows a rear view of the image distance control device of FIG. 34 with the extendable portion in the extended position;

FIG. 42 shows a front view of the image distance control device of FIG. 34 with a cellular telephone secured in place therein;

FIG. 43 shows a side view of the image distance control device of FIG. 34 with a cellular telephone secured in place therein;

FIG. 44 shows a perspective view of the image distance control device of FIG. 34 with the extendable portion in a closed position; and

FIG. 45 shows a perspective view of the image distance control device of FIG. 34 with the extendable portion in the extended position.

### DETAILED DESCRIPTION

In accordance with embodiment, an apparatus for measuring a distance between an imaging device and a wound is provided. The device includes a photographic device holder portion, a distance measurement device, and a light source.

As used herein, the terms “cellular telephone” and “cell phone” are used interchangeably. As used herein, the term “photographic device” is any device capable of capturing a photographic image of an object. A photographic device may include, without limitation, a camera, a cellular telephone, any device having a camera, etc.

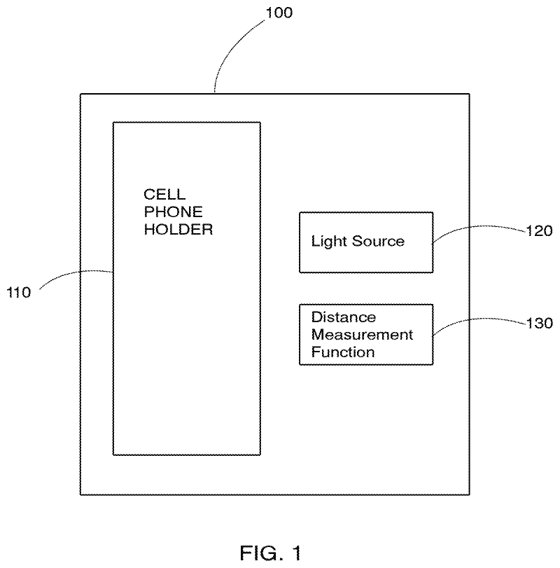

FIG. 1 shows functional components of an image distance control device in accordance with an embodiment. Image distance control device 100 includes a photographic device holder 110, also referred to as cell phone holder 110, a light source 120, and a distance measurement function 130. Photographic device holder 110 includes a mechanism for supporting a photographic device, such as a cellular telephone, in a selected position while a user employs the cell phone to capture a photograph. Photographic device holder 110 is adapted to hold the photographic device (e.g., the cell phone) in a position that enables the user to view the viewing screen of the photographic device while ensuring that the camera function of the photographic device (including, for example, the camera lens of a cell phone) has an unimpeded view of the wound to be imaged. Light source 120 provides light to illuminate the subject of the photograph. For example, light source 120 may illuminate a wound or injury while a user employs a cell phone to take a photograph of the wound or injury. Distance measurement function 130 determines a distance from image control device 100 to a selected surface or object. For example, distance measurement function 130 may determine a distance between image distance control device 100 and a wound or injury which a user wishes to photograph.

Accordingly, in one embodiment, supposing that a user wishes to employ image distance control device 100 to capture an image of a wound on a patient's body, the user may secure a cellular telephone that has a camera in photographic device holder 110. The user positions image distance control device 100 to ensure that the camera of the cell phone is positioned to capture an image of the wound. The user employs distance measurement function 130 to verify that the distance between image distance control device 100 and the wound is at a desired distance. In one embodiment, the user may specify a predetermined distance or range. Image distance control device 100 may provide a visible indication, such as a light, or an audible indication, such as a beep or other sound, when the determined distance is equal to the predetermined distance or within the predetermined range. When the user determines that the distance is correct, the user employs the cell phone to take a photograph of the wound.

Distance measurement function 130 may also have the capability to determine an angle formed between image distance control device 100 and a surface that contains the wound to be photographed. An indicator may notify the user when the angle is equal to a specified angle.

Image distance control device 100 may include a switch or button to control light source 120. For example, in a conventional manner, a user may push a button to turn light source 120 on and off.

In one embodiment, a laser device is used to measure distance. In another embodiment, the distance measurement device includes an infrared distance measurement device.

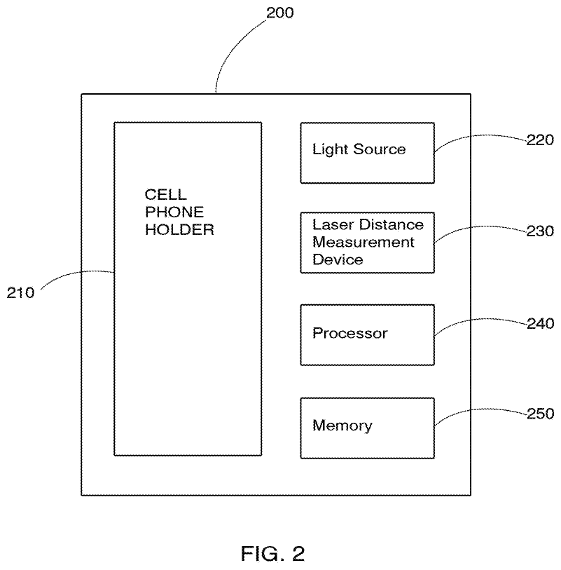

FIG. 2 shows functional components of an image distance control device in accordance with another embodiment. Image distance control device 200 includes a photographic device holder 210 (shown as cell phone holder 210), a light source 220, a laser distance measurement device 230, a processor 240, and a memory 250. Photographic device holder 210 includes a mechanism for supporting a photographic device, such as a cellular telephone, in a selected position while a user employs the photographic device to capture a photograph. Light source 220 provides light to illuminate the subject of the photograph. For example, light source 220 may include, for example, one or more incandescent light sources, one or more fluorescent lights, or light emitting diode (LED) devices. Other types of light sources may be used. Laser distance measurement device 230 includes a laser device adapted to direct a laser at a surface or object and a detector to detect reflected laser light. Processor 240 includes software and/or hardware adapted to determine a distance between the image distance control device 200 and the surface or object based on the detected laser light. Memory 250 is adapted to store data and is used by processor 240 to store data and other information. Processor 240 may control the operation of laser distance measurement device 230 and/or light source 220. In another embodiment, an infrared distance measurement device is used. Other types of distance measurement devices may be used.

In one embodiment, laser distance measurement device 230 additionally verifies that an angle formed by the laser signal and the surface containing the wound is a desired angle. For example, laser distance measurement device 230 may verify that the laser directed at the surface containing the wound is perpendicular to the surface (i.e., forms an angle of ninety degrees relative to the surface).

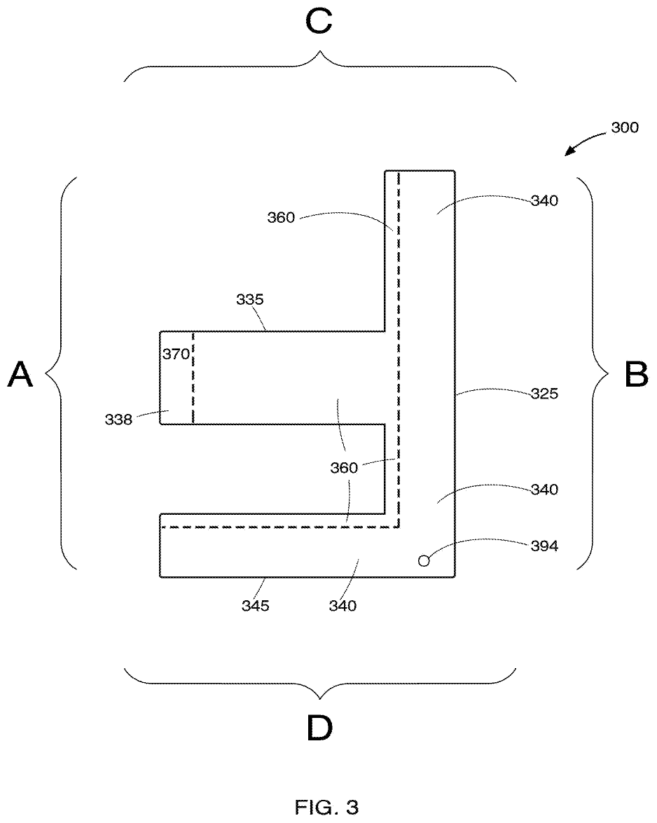

FIG. 3 shows a front view of an image distance control device in accordance with an embodiment. Image distance control device 300 includes a stem section 325, a middle arm section 335, and a lower arm section 345. Image distance control device 300 may be formed of plastic, metal, or another suitable material.

In FIG. 3, a dotted line is used to show a change of surface level. Thus, a first region 360 having a lower level covers portions of stem section 325, middle arm section 335, and lower arm section 345. A raised region 340 covers portions of stem section 325 and lower arm section 345. A projecting element 338 having a raised region 370 is disposed at the end of middle arm section 335.

The varying surface levels of image distance control device 300 allow a portion of the device to function as a photographic device holder. In particular, the regions of image distance control device 300 that have a surface at a lower level, such as region 360, are adapted to hold a photographic device such as a cellular telephone. Thus, in one embodiment, a cellular telephone having a camera function may be placed into region 360 and is held in place by the sides associated with the higher-level surfaces situated around region 360, such as region 340 and region 370. While resting in region 360, the camera lens of the cellular telephone has an unimpeded view (i.e., the camera lens is not blocked by stem section 325, middle arm section 335 or lower arm section 345) and may capture images of a particular wound or other object.

In one embodiment, projecting element 338 is adapted to move relative to middle arm section 335 (e.g., slide in and out relative to middle arm section 335) to allow a user to expand the space available for holding a cellular telephone. For example, projecting element 338 may slide up to one inch away from middle arm section 335. This feature allows the device to adjust the size of the cell phone holding space in order to accommodate various sizes of cell phones.

While the illustrative embodiment shows an image distance control device adapted to hold a cellular telephone, in other embodiments, an image distance control device may be adapted to hold other types of photographic devices.

An opening 394 contains a lens through which a user may view a wound that is to be imaged. Opening 394 may be one side of a hole that passes through the image distance control device 300. In one embodiment, laser distance measurement device 230 (not shown in FIG. 3) indicates to a user when the distance between image distance control device 300 and a wound to be imaged equals a specified distance. For example, laser distance measurement device 230 may cause an indication, such as a red dot, to be overlaid on an image of a wound when the distance between the image distance control device and the wound equals a specified distance. If the distance does not equal the specified distance, the indicator is not displayed.

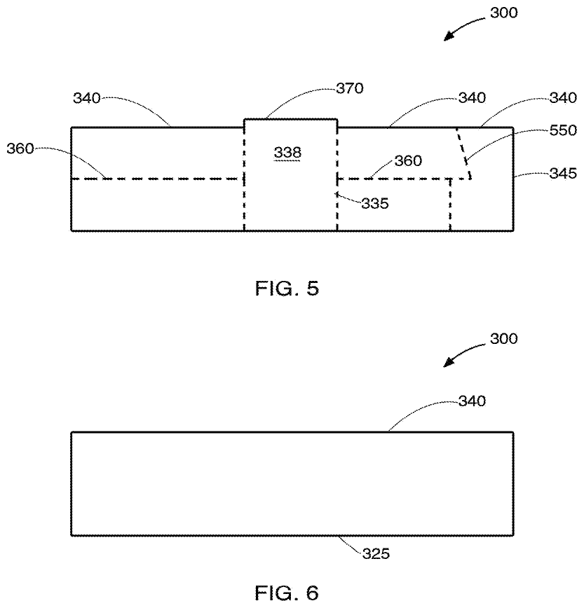

As indicated in FIG. 3, the four sides of image distance control device 300 are referred to herein and in the Figures as sides A, B, C, and D. Sides A, B, C, and D are shown in FIGS. 5, 6, 7, and 8, respectively.

In various embodiments, image distance control device 300 may be between 4 and 8 inches high and between 3 and 6 inches wide. Other dimensions may be used.

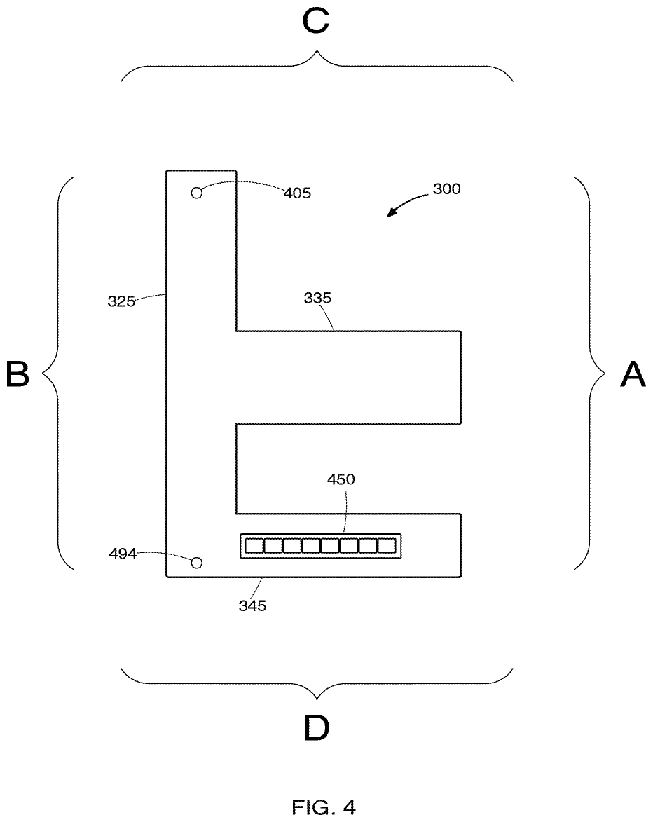

FIG. 4 shows a rear view of the image distance control device of FIG. 3. Stem section 325 includes an opening 405 that is associated with a laser distance measurement device (not shown) disposed inside stem section 325. Thus, the laser distance measurement device may project a laser through opening 405 toward a selected object or surface. A light source 450 is disposed on a surface of lower arm section 345. Light source 450 may be, for example, an incandescent light bulb, a fluorescent light source, one or more LED lights, or another type of light source. A processor and memory (not shown) may also be disposed within stem section 325 or within lower arm section 345. The processor may control the operation of the laser distance measurement device and the light source. Opening 494 is the opposite end of opening 394 (shown in FIG. 3), and allows a user to view a wound that is to be photographed.

FIG. 5 shows side A of the image distance control device of FIG. 3. Lower arm section 345 has an inner surface 550 that joins surface 340 and surface 360. Surface 550 and surface 360 form an acute angle. The acute angle allows a cell phone to be securely held in place within image distance control device 300.

FIG. 6 shows side B of the image distance control device of FIG. 3.

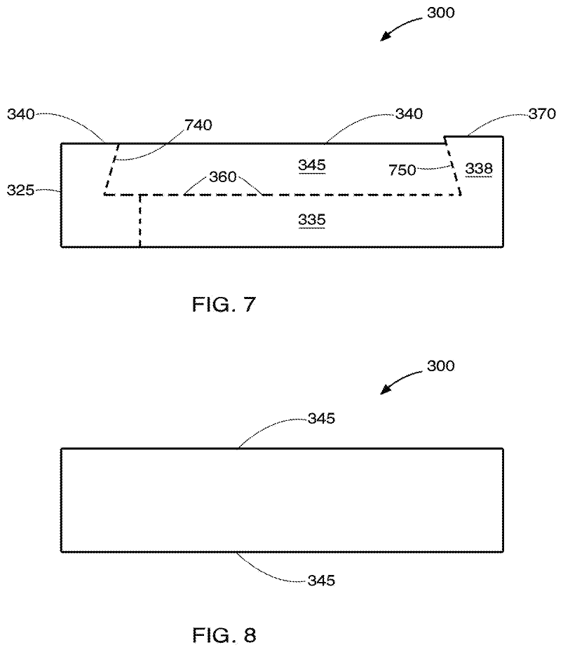

FIG. 7 shows side C of the image distance control device of FIG. 3. Stem section 325 includes a surface 740 that joins surface 340 and surface 360. Surface 740 and surface 360 form an acute angle. Similarly, projecting element 338 includes a surface 750 that joins surface 370 and surface 360. Surface 750 and surface 360 form an acute angle. The acute angles allow a cell phone to be securely held in place within image distance control device 300.

FIG. 8 shows side D of the image distance control device of FIG. 3.

In accordance with an embodiment, a photographic device, such as a cell phone, is placed in and secured within an image distance control device. Once the photographic device is secured in the device, the image distance control device is used to verify that the photographic device is at a predetermined distance from (and at a desired angle relative to) a wound on a patient's body. A user then employs the camera function of the photographic device (e.g., the cell phone) to generate a photograph of the wound. The image distance control device may provide a desired level of lighting for the photograph.

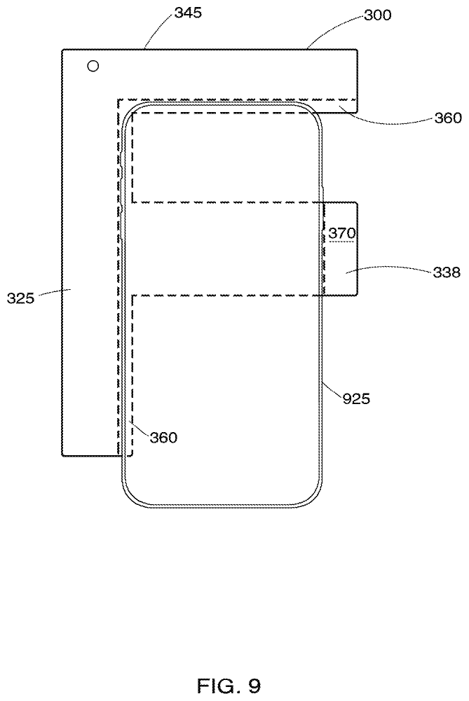

FIG. 9 shows a front view of a cell phone secured in the image distance control device of FIG. 3. Specifically, a cell phone 925 having a camera function is disposed in image distance control device 300. Various portions of image distance control device 300, including stem section 325, lower arm section 345, and projecting element 338, hold cell phone 925 in place. The camera of cell phone 925 may be disposed on the side of cell phone 925 opposite the side shown in FIG. 9 (and therefore is not visible in FIG. 9).

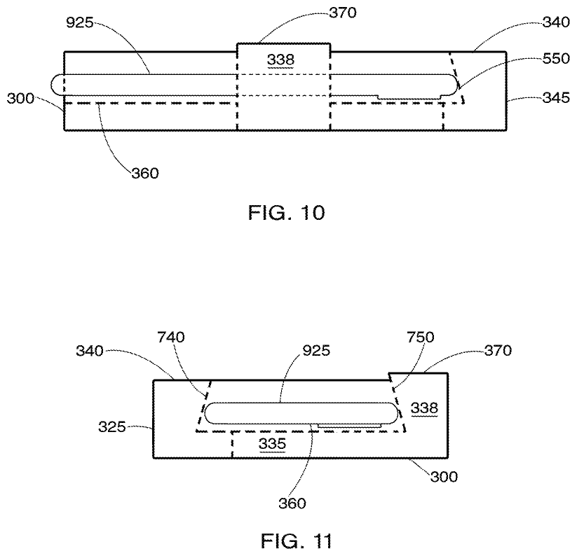

FIG. 10 shows a side view of cell phone 925 secured in image distance control device 300 (seen from side A of image distance control device 300). Cell phone 925 rests on surface 360, and is secured in place by angled surfaces such as surface 550 of lower arm section 345.

FIG. 11 shows another side view of cell phone 925 secured in image distance control device 300 (seen from side C of image distance control device 300). Cell phone 925 rests on surface 360, and is secured in place by angled surfaces such as surfaces 740 and 750.

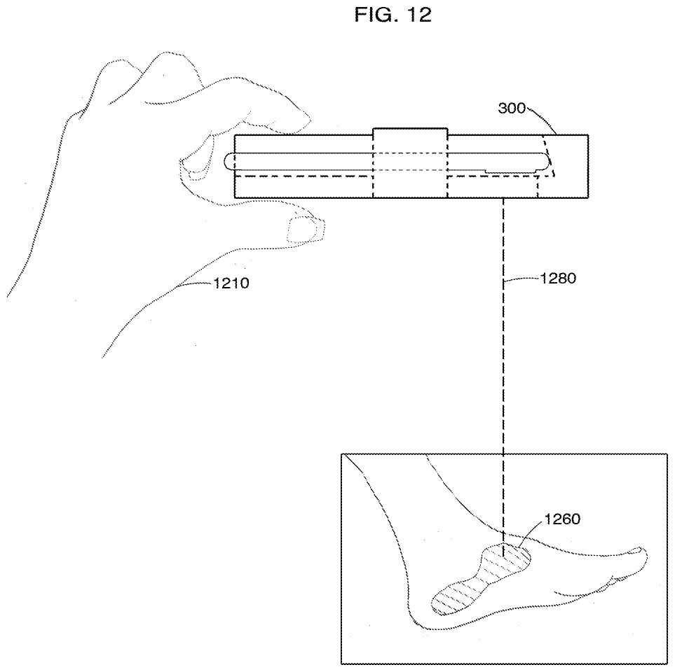

In accordance with an embodiment, an image distance control device may be employed to determine and verify a distance between the device and a wound that is to be imaged. The device may then be used to hold a photographic device, such as a cell phone, while a user employs a camera function of the cell phone to capture a photograph of the wound. FIG. 12 shows an image distance control device being used to verify a distance to a wound to be imaged in accordance with an embodiment. Image distance control device 300 is held in a user's hand 1210 above a wound 1260 located on a part of a patient's body. Image distance control device 300 holds a cell phone. Image distance control device 300 directs a laser signal 1280 at the wound and detects the reflected signal. Image distance control device 300 determines the distance to the wound based on the reflected signal. After the user verifies that the image distance control device 300 is located at a desired distance (and angle) from the wound, the user may employ the cell phone to capture an image of the wound 1260.

In one embodiment, the image distance control device is configured to ensure that all images of a wound are taken at twelve (12) inches distance. Thus, image distance control device 300 is configured to verify and indicate that the distance between the device and the wound is correct when the distance between the device and the wound is determined to be twelve (12) inches. In another embodiment, the device may indicate that the distance is correct when the distance between the device and the wound is determined to be a first predetermined distance (e.g., 12 inches) plus or minus a second predetermined distance, which provides a margin of error for the user (e.g., one-half inch). Other distances may be used. In another embodiment, the image control device also verifies that the angle between the image distance control device and the wound is 90 degrees. Other angles may be used.

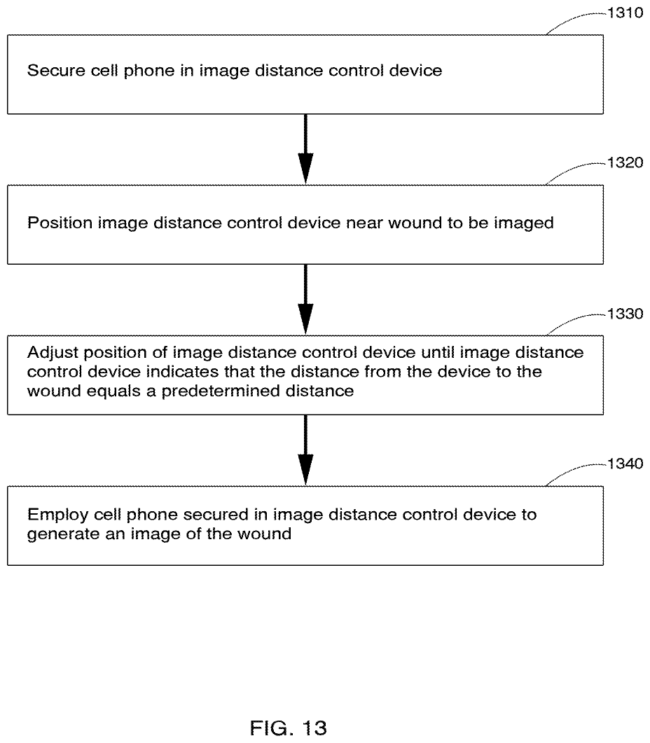

FIG. 13 is a flowchart of a method of using an image distance control device to determine a distance in accordance with an embodiment. At step 1310, a cell phone (or other photographic device) is secured in an image distance control device. At step 1320, the image distance control device is positioned near a wound that is to be imaged. At step 1330, the position of the image distance control device is adjusted until the image distance control device indicates that the distance from the device to the wound equals a predetermined distance. At step 1340, the cell phone (or other photographic device) secured in the image distance control device is employed to generate an image of the wound.

In one embodiment, an image distance control device may including a printing function/capability. After an image of a wound is captured, the device may transmit the image, or cause the image to be transmitted, to a printer device and cause the printer device to print the image. For example, the image distance control device may be in communication with the photographic device secured therein. The connection to the printer device may be via a wired connection or may be via a wireless connection.

In various embodiments, the method steps described herein, including the method steps described in FIG. 13, may be performed in an order different from the particular order described or shown. In other embodiments, other steps may be provided, or steps may be eliminated, from the described methods.

Systems, apparatus, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be used within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc.

Systems, apparatus, and methods described herein may be implemented using a computer program product tangibly embodied in an information carrier, e.g., in a non-transitory machine-readable storage device, for execution by a programmable processor; and the method steps described herein, including one or more of the steps of FIG. 13, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

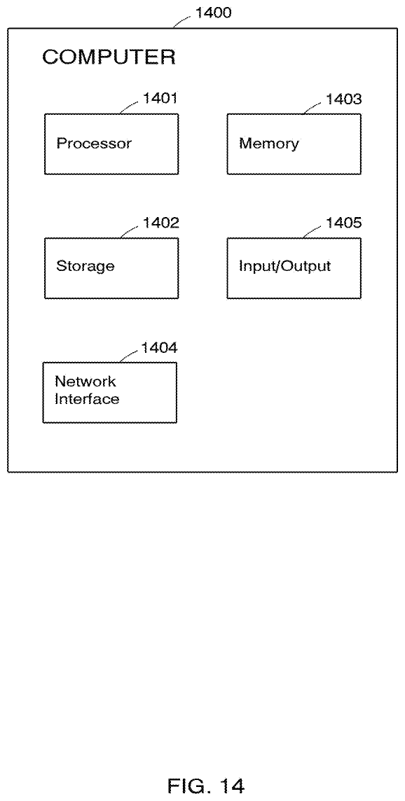

A high-level block diagram of an exemplary computer that may be used to implement systems, apparatus and methods described herein is illustrated in FIG. 14. Computer 1400 includes a processor 1401 operatively coupled to a data storage device 1402 and a memory 1403. Processor 1401 controls the overall operation of computer 1400 by executing computer program instructions that define such operations. The computer program instructions may be stored in data storage device 1402, or other computer readable medium, and loaded into memory 1403 when execution of the computer program instructions is desired. Thus, the method steps of FIG. 13 can be defined by the computer program instructions stored in memory 1403 and/or data storage device 1402 and controlled by the processor 1401 executing the computer program instructions. For example, the computer program instructions can be implemented as computer executable code programmed by one skilled in the art to perform an algorithm defined by the method steps of FIG. 13. Accordingly, by executing the computer program instructions, the processor 1401 executes an algorithm defined by the method steps of 13. Computer 1400 also includes one or more network interfaces 1404 for communicating with other devices via a network. Computer 1400 also includes one or more input/output devices 1405 that enable user interaction with computer 1400 (e.g., display, keyboard, mouse, speakers, buttons, etc.).

Processor 1401 may include both general and special purpose microprocessors, and may be the sole processor or one of multiple processors of computer 1400. Processor 1401 may include one or more central processing units (CPUs), for example. Processor 1401, data storage device 1402, and/or memory 1403 may include, be supplemented by, or incorporated in, one or more application-specific integrated circuits (ASICs) and/or one or more field programmable gate arrays (FPGAs).

Data storage device 1402 and memory 1403 each include a tangible non-transitory computer readable storage medium. Data storage device 1402, and memory 1403, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1405 may include peripherals, such as a printer, scanner, display screen, etc. For example, input/output devices 1405 may include a display device such as a cathode ray tube (CRT) or liquid crystal display (LCD) monitor for displaying information to the user, a keyboard, and a pointing device such as a mouse or a trackball by which the user can provide input to computer 1400.

One skilled in the art will recognize that an implementation of an actual computer or computer system may have other structures and may contain other components as well, and that FIG. 14 is a high level representation of some of the components of such a computer for illustrative purposes.

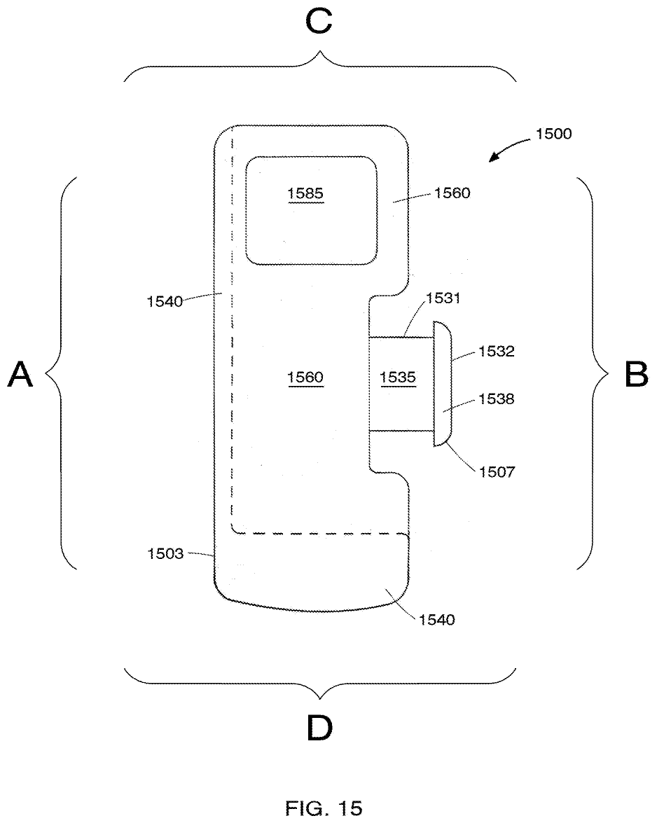

FIGS. 15-21 show an image distance control device in accordance with another embodiment. Specifically, FIG. 15 shows a front view of an image distance control device 1500.

In FIG. 15, a dotted line is used to show a change of surface level. Thus, image distance control device 1500 includes a first region 1560 having a first, lower level, and a second region 1540 having a second, higher level.

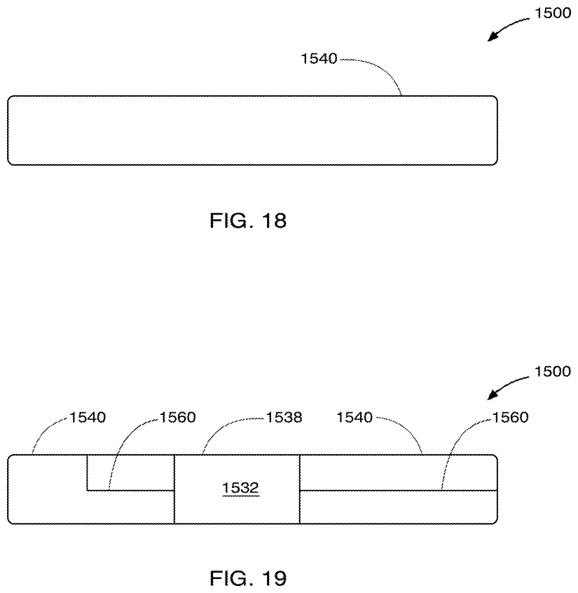

As indicated in FIG. 15, the four sides of image distance control device 1500 are referred to herein and in the Figures as sides A, B, C, and D. Sides A, B, C, and D are shown in FIGS. 18, 19, 20, and 21, respectively.

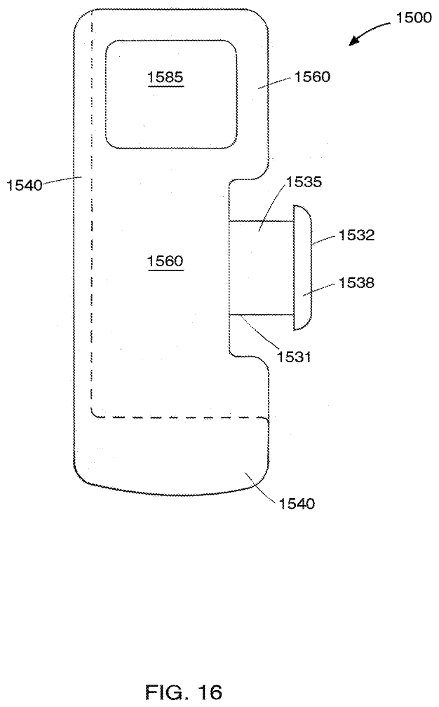

Image distance control device 1500 includes a main body section 1503 (comprising regions 1540 and 1560) and an extendable arm section 1507 that includes an arm section 1531 and an end section 1532. A top surface of arm section 1531 forms a region 1535 having a level flush with or substantially flush with the level of region 1560, so that regions 1560 and 1535 form a substantially continuous surface. A top surface of end section 1532 forms a region 1538 that is raised relative to region 1535 and which has a level that is at the same or substantially the same height as region 1540. The extendable arm section 1507 is adapted to slide in and out of the main body section 1503 of the device to accommodate various cellular telephones of different sizes. Extendable arm section 1507 has a non-extended position and a (fully) extended position, or may occupy a partially extended position between the non-extended position and the (fully) extended position. FIG. 16 shows image distance control device 1500 with the extendable arm section 1507 in the extended position in accordance with an embodiment.

Regions 1540, 1560, 1535, and 1538 are adapted to hold a cellular telephone in a secure position to enable a user to position the cell phone and use the cell phone's camera function to capture an image of a selected object (such as a wound).

Image distance control device 1500 includes an opening 1585 disposed in region 1560. Opening 1585 is located suitably to allow a camera lens of a cell phone placed in region 1560 to have an unobstructed view of a selected object and to be able to capture an image of the object.

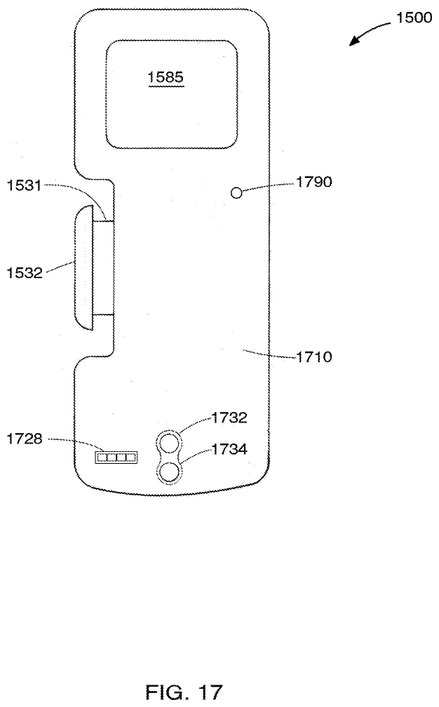

FIG. 17 shows a rear view of image distance control device 1500. The rear side of image distance control device 1500 includes a surface 1710, a lighting element 1728, and two buttons 1732 and 1734 adapted to control lighting element 1728. For example, buttons 1732, 1734 may turn lighting element 1728 on and off, and/or may increase or decrease the intensity of lighting element 1728.

Also located on the rear side of image distance control device 1500 is an opening 1790 associated with a laser distance measurement device disposed inside device 1500. Thus, the laser distance measurement device may project a laser through opening 1790 toward a selected object or surface.

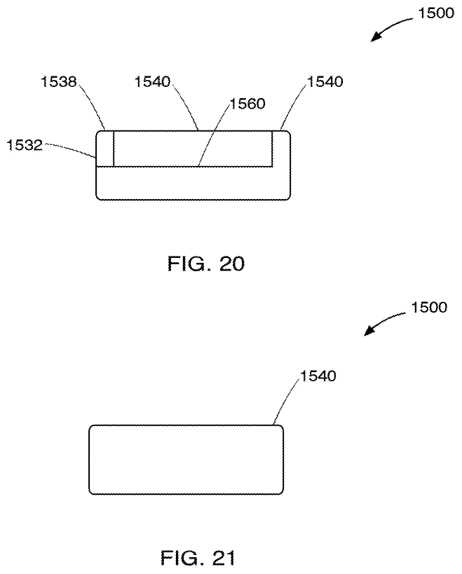

FIG. 18 shows a view of side A of image distance control device 1500. FIG. 19 shows a view of side B of image distance control device 1500. FIG. 20 shows a view of side C of image distance control device 1500. FIG. 21 shows a view of side D of image distance control device 1500.

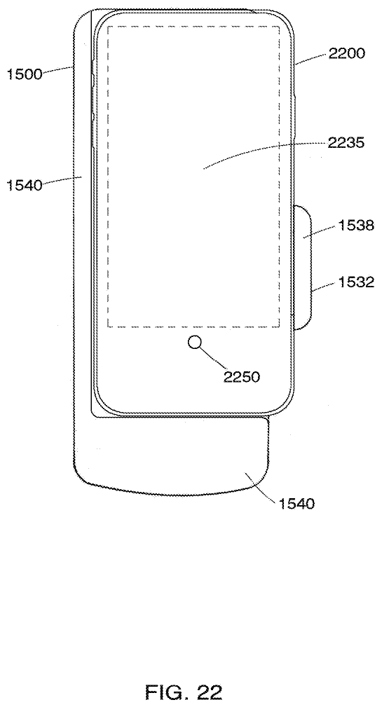

FIG. 22 shows a front view of image distance control device 1500 with a cellular telephone secured therein in accordance with an embodiment. Cellular telephone 2200 is disposed in region 1560 and is held in place by raised regions 1540 and 1538.

In the illustrative embodiment, the camera function of cellular telephone 2200 is activated. Accordingly, a field of view 2235 is displayed on the screen of cellular telephone 2200 and a button 2250 for controlling the camera function is displayed on the screen.

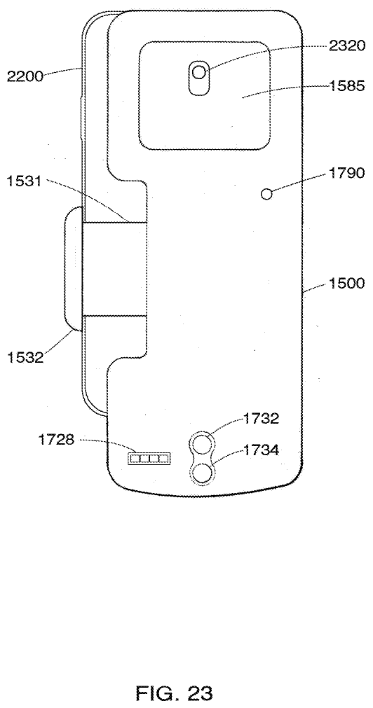

FIG. 23 shows a rear view of image distance control device 1500 with cellular telephone 1500 secured therein. A camera 2320 of telephone 2200 has an unobstructed view through opening 1585.

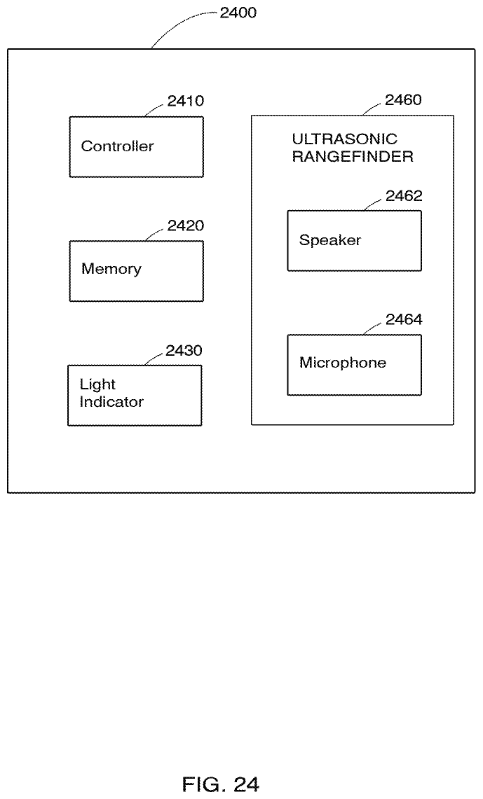

In accordance with another embodiment, a user may employ an ultrasonic rangefinder device to determine a desired distance to a selected surface, such as a surface of a wound, and use a camera to capture an image of the surface from the desired distance. FIG. 24 shows components of an ultrasonic rangefinder device in accordance with an embodiment. Ultrasonic rangefinder device 2400 includes a controller 2410, a memory 2420, and an ultrasonic rangefinder component 2460. Ultrasonic rangefinder component 2460 includes a speaker 2462 and a microphone 2464. The operation of ultrasonic rangefinders is known. Ultrasonic rangefinder component 2460 may determine distance between the device and a selected surface, such as the surface of a wound. Ultrasonic rangefinder device 2400 also includes a light indicator 2430. Light indicator 2430 may display a light, for example, when the determined distance equals a predetermined distance, for example, or when the determined distance is within a predetermined range.

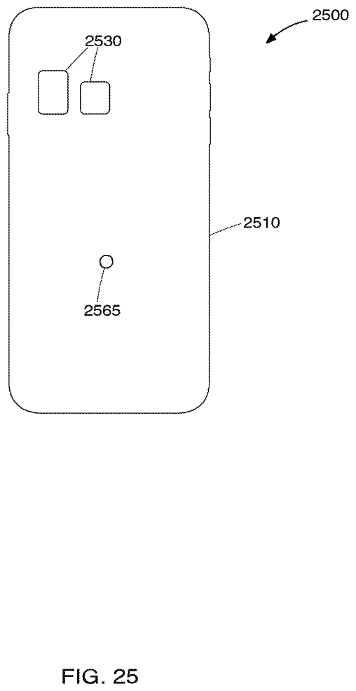

FIG. 25 shows an example of an ultrasonic rangefinder device in accordance with an embodiment. Ultrasonic rangefinder device 2500 includes a housing 2510 which contains the components of the ultrasonic rangefinder and several buttons 2530 which may be used to control the operation of the ultrasonic rangefinder device 2500. Ultrasonic rangefinder 2500 also includes a light indicator 2565, which may turn on when the device detects that a distance to a selected surface is equal to a predetermined distance or falls within a predetermined range.

A user may employ ultrasonic rangefinder device 2500 to determine a desired distance to a patient's wound, and then use a camera device (such as a cell phone) to capture an image from the desired distance.

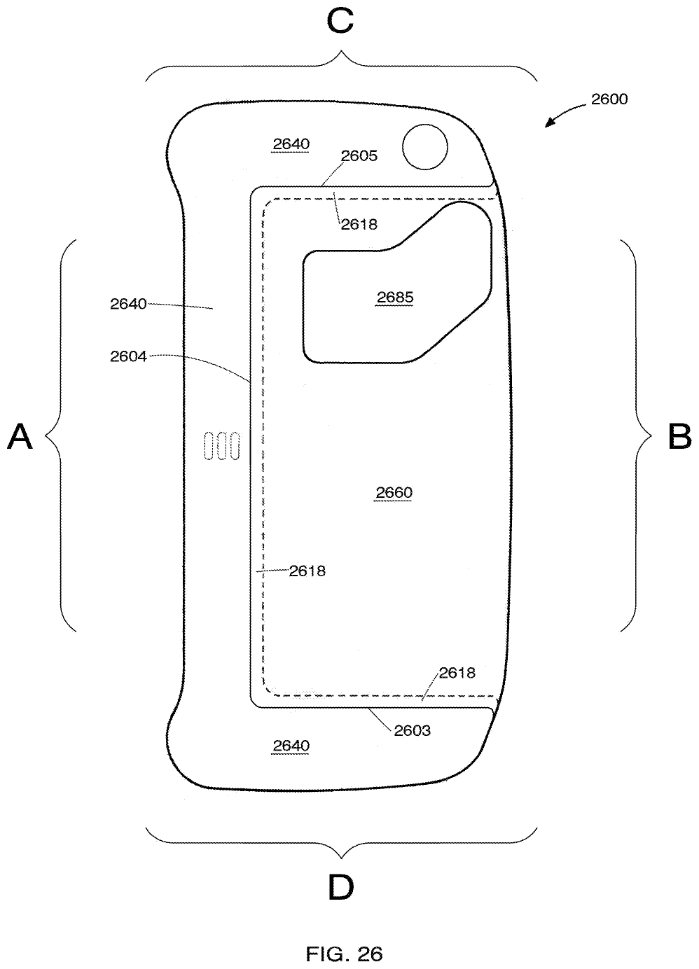

FIGS. 26-33 show an image distance control device in accordance with another embodiment. Specifically, FIG. 26 shows a front view of an image distance control device 2600.

In FIG. 26, a dotted line is used to show a change of surface level. Thus, image distance control device 2600 includes a first region 2660 having a first, lower level, and a second region 2640 having a second, higher level. A material 2618 is disposed at one or more edges or boundaries of first region 2660. In the illustrative embodiment, boundaries of region 2640 are defined by walls 2603, 2604, and 2605. A layer of material 2618 is disposed adjacent to, and may be attached to, walls 2603, 2604, 2605. Thus, material 2618 is disposed between walls 2603, 2604, 2605 and region 2660. Material 2618 functions to hold a cell phone or other device in region 2660. For example, material 2618 may include an elastomer, a plastic, or other material. When a cell phone is placed in region 2660, portions of material 2618 that come into contact with the cell phone may contract to accommodate the device; as a result, the material may press on the edges of the device and hold it in place.

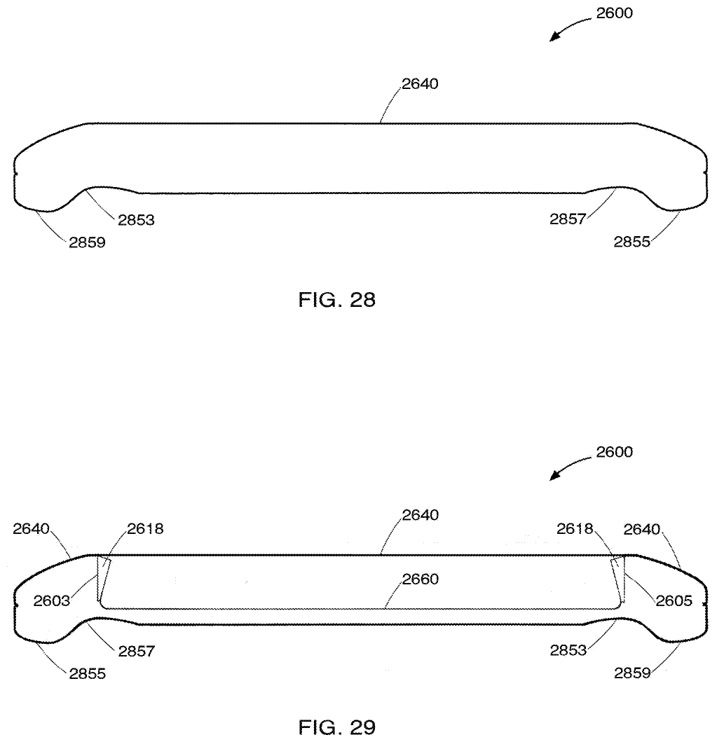

As indicated in FIG. 26, the four sides of image distance control device 2600 are referred to herein and in the Figures as sides A, B, C, and D. Sides A, B, C, and D are shown in FIGS. 28, 29, 30, and 31, respectively.

Regions 2640 and 2660 are adapted to hold a cellular telephone (or other device) in a secure position to enable a user to position the cell phone and use the cell phone's camera function to capture an image of a selected object (such as a wound).

Image distance control device 2600 includes an opening 2685 disposed in region 2660. Opening 2685 is located suitably to allow a camera lens of a cell phone placed in region 2660 to have an unobstructed view of a selected object and to be able to capture an image of the object.

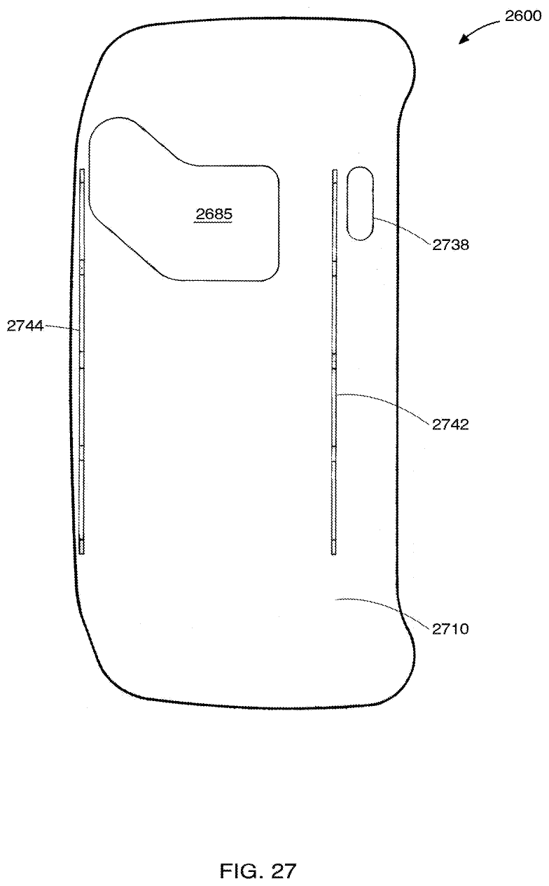

FIG. 27 shows a rear view of image distance control device 2600. The rear side of image distance control device 2600 includes a surface 2710, lighting elements 2742, 2744, and an aperture or window 2738 associated with a distance measurement device, which may be an infrared distance measurement device or a laser distance measurement device, for example.

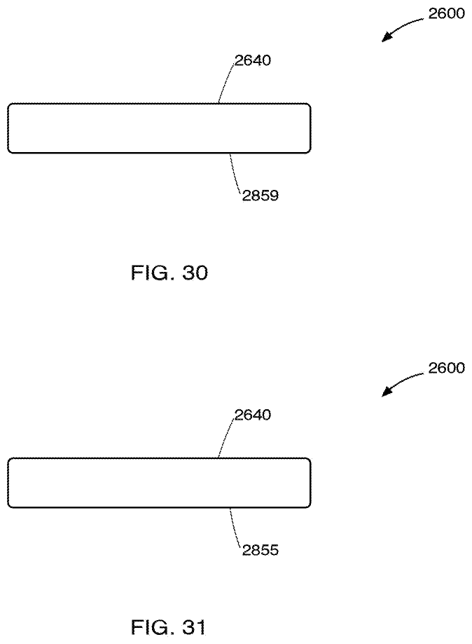

FIG. 28 shows a view of side A of image distance control device 2600. FIG. 29 shows a view of side B of image distance control device 2600. FIG. 30 shows a view of side C of image distance control device 2600. FIG. 31 shows a view of side D of image distance control device 2600.

Referring to FIG. 28, image distance control device 2600 has a shape and design that facilitate a user's holding and handling thereof. Specifically, the ends of image distance control device 2600 associated with sides C and D include projections 2859 and 2855, respectively, that project outwardly from the rear side of the device. Proximate projection 2855 is an indentation 2857, and proximate projection 2859 is an indentation 2853. The indentations and projections may make it easier for a user to manipulate the device, and to hold the device without inadvertently dropping it, for example. In one embodiment, each indentation 2853, 2857 has a cross-sectional shape that defines a portion of the circumference of a circle. Other shapes may be used.

Referring to FIG. 29, material 2618 is attached to walls 2603 and 2605. Material 2618 may also be attached to wall 2604 (not shown). Material 2618 forms an acute angle with respect to the surface of region 2660. In particular, the thickness of material 2618 increases as a function of distance from the surface of region 2660; therefore, the portion of material 2618 that is farther from the surface of region 2660 is thicker and consequently projects further from walls 2603, 2604, 2605 than the portion of material 2618 that is closer to the surface of region 2660. For example, material 2618 may have a triangular cross-section, as shown in FIG. 29. The resulting shape helps to hold a cellular telephone in place when the cellular telephone is placed into region 2660.

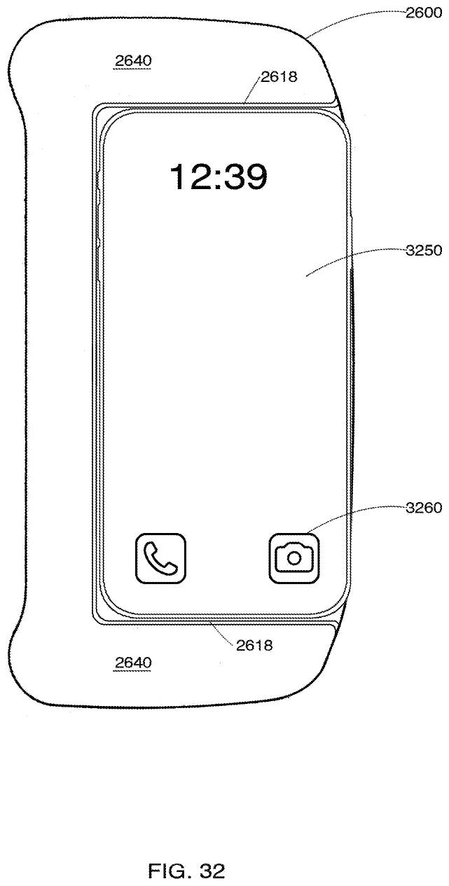

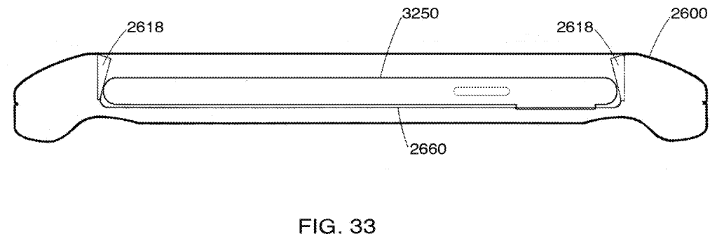

FIG. 32 shows a front view of the image distance control device of FIG. 26 with a cellular telephone secured in place therein. Specifically, FIG. 32 shows a cellular telephone 3250 disposed in image distance control device 2600. A camera icon 3260, which may be used to access a camera function, is displayed on the display screen of cellular telephone 3250. FIG. 33 shows a side view of image distance control device 2600 with cellular telephone 3250 disposed therein.

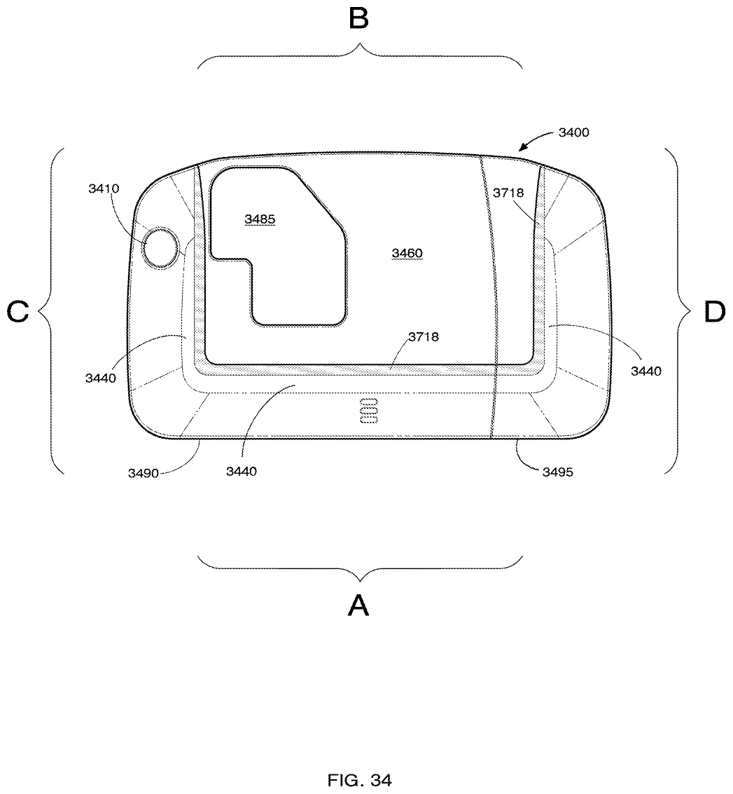

FIGS. 34-45 show an image distance control device in accordance with another embodiment. Specifically, FIG. 34 shows a front view of an image distance control device 3400.

Image distance control device 3400 includes a first region 3460 having a first, lower level, and a second region 3440 having a second, higher level. A layer of material 3718 is disposed on one or more edges of region 3460. Material 3718 may be an elastomer, a plastic, or other material.

Regions 3460 and 3440 are adapted to hold a cellular telephone (or other device) in a secure position to enable a user to position the cell phone and use the cell phone's camera function to capture an image of a selected object (such as a wound).

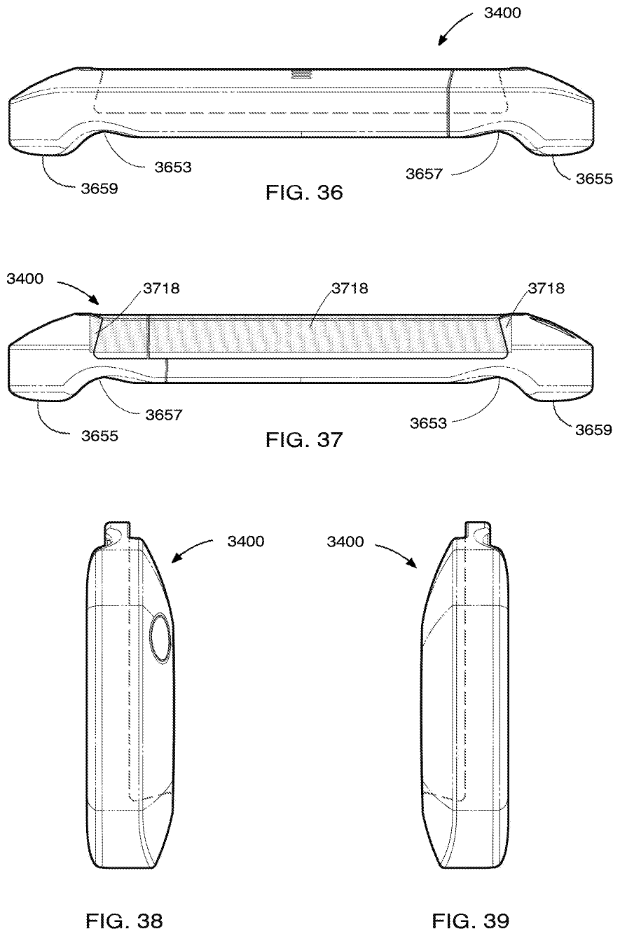

As indicated in FIG. 34, the four sides of image distance control device 3400 are referred to herein and in the Figures as sides A, B, C, and D. Sides A, B, C, and D are shown in FIGS. 36, 37, 38, and 39, respectively.

Image distance control device 3400 includes an opening 3485 disposed in region 3460. Opening 3485 is located suitably to allow a camera lens of a cell phone (or other photographic device) placed in region 3460 to have an unobstructed view of a selected object and to be able to capture an image of the object.

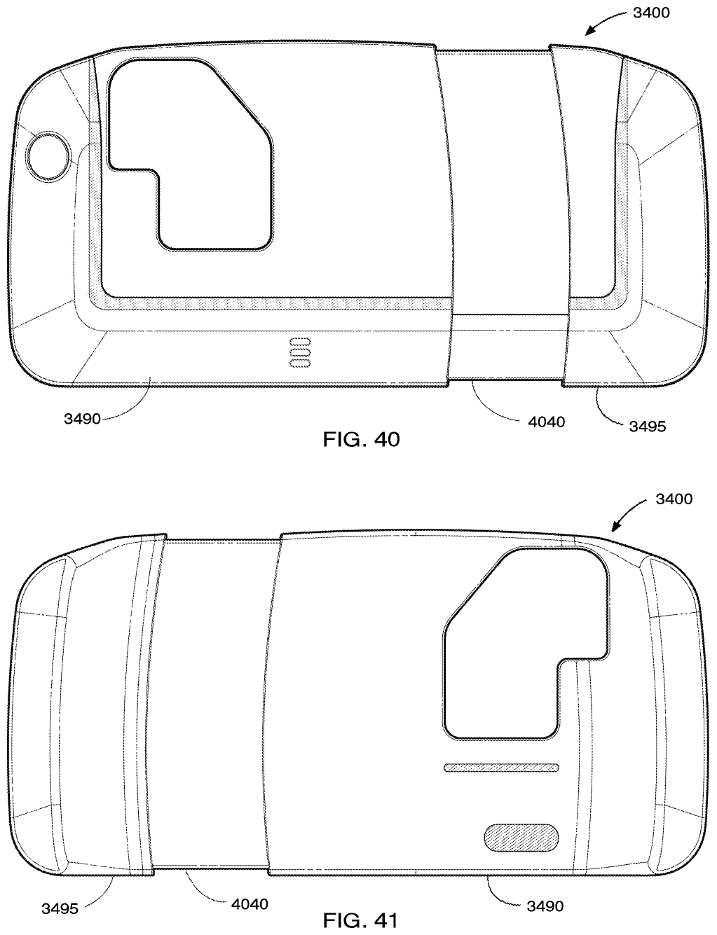

Image distance control device 3400 includes a fixed portion and an extendable portion that is adapted to slide in and out of a slot or cavity in the fixed portion. In the illustrative embodiment of FIG. 34, the fixed portion includes a first part 3490 and the extendable portion includes a second part 3495. The extendable portion may extended in order to adjust the length of the device to accommodate different sizes of cell phones. Thus, moving the extendable portion (including second part 3495) from the closed position to the extended position increases the surface area of first region 3460, enabling image distance control device 3400 to accommodate larger sizes of cellular telephones. Second part 3495 is adapted to move between a first, closed position (shown in FIG. 34) in which it contacts first part 3490, and a second, extended position, in which it is separated from first part 3490. The second, extended position is illustrated in FIG. 40.

Image distance control device 3400 includes a button 3410 which is a power on/power off switch.

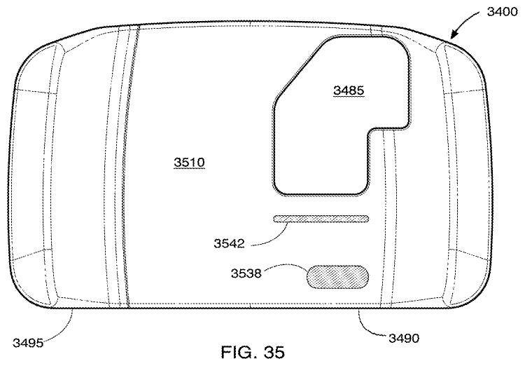

FIG. 35 shows a rear view of image distance control device 3400 in accordance with an embodiment. The rear side of image distance control device 3400 includes a surface 3510, a lighting element 3542, and an aperture or window 3538 of a distance measurement device. For example, lighting element 3542 may be an LED lighting device, or may be another type of lighting device. Window 3538 may be a window of a laser distance measurement device, an infrared (IR) distance measurement device, or part of another distance measurement device.

FIG. 36 shows a view of side A of image distance control device 3400. FIG. 37 shows a view of side B of image distance control device 3400. FIG. 38 shows a view of side C of image distance control device 3400. FIG. 39 shows a view of side D of image distance control device 3400.

Referring again to FIG. 36, image distance control device 3400 has a shape and design that facilitate a user's holding and handling thereof. Specifically, the top and bottom ends of image distance control device 3400 (proximate sides C and D, respectively) include projections 3659 and 3655, respectively, which project outwardly on the rear side of the device. Proximate projection 3659 is an indentation 3653, and proximate projection 3655 is an indentation 3657. The indentations and projections may make it easier for a user to manipulate the device, and to hold the device without inadvertently dropping it, for example. In one embodiment, each indentation 3653, 3657 may have a cross-sectional shape that defines a portion of the circumference of a circle. Other shapes may be used.

FIG. 40 shows image distance control device 3400 with the extendable portion (including second part 3495) in the second, extended position in accordance with an embodiment. The extendable portion also includes a section 4040 attached to second part 3495. Section 4040 is disposed between first part 3490 and second part 3495 and connects first part 3490 and second part 3495. Section 4040 is adapted to slide into and out of a slot or cavity within first part 3490.

FIG. 41 shows a rear view of image distance control device 3400 with second part 3495 in the second, extended position in accordance with an embodiment.

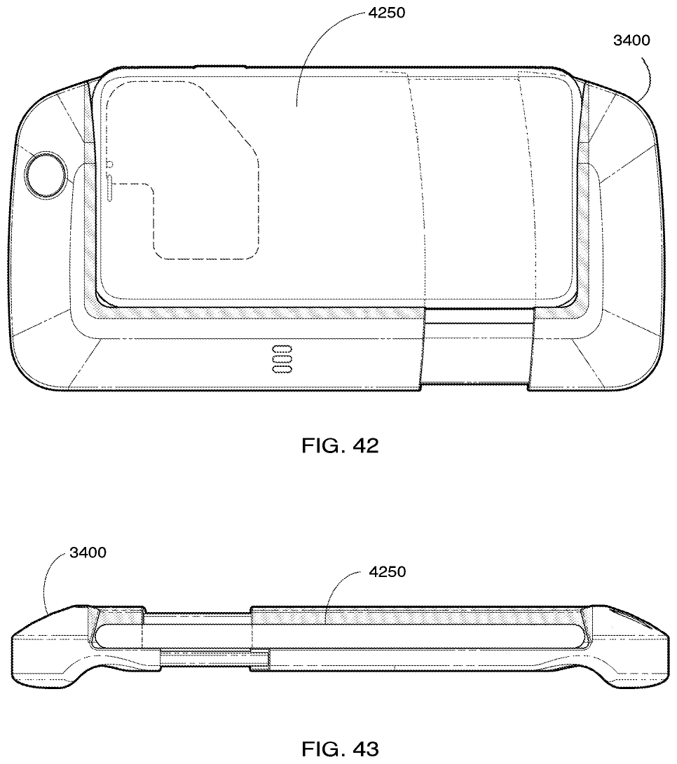

FIG. 42 shows a front view of an image distance control device with a cellular telephone secured in place therein in accordance with an embodiment. Specifically, FIG. 42 shows a cellular telephone 4250 disposed in image distance control device 3400. FIG. 43 shows a side view of image distance control device 3400 with cellular telephone 4250 disposed therein.

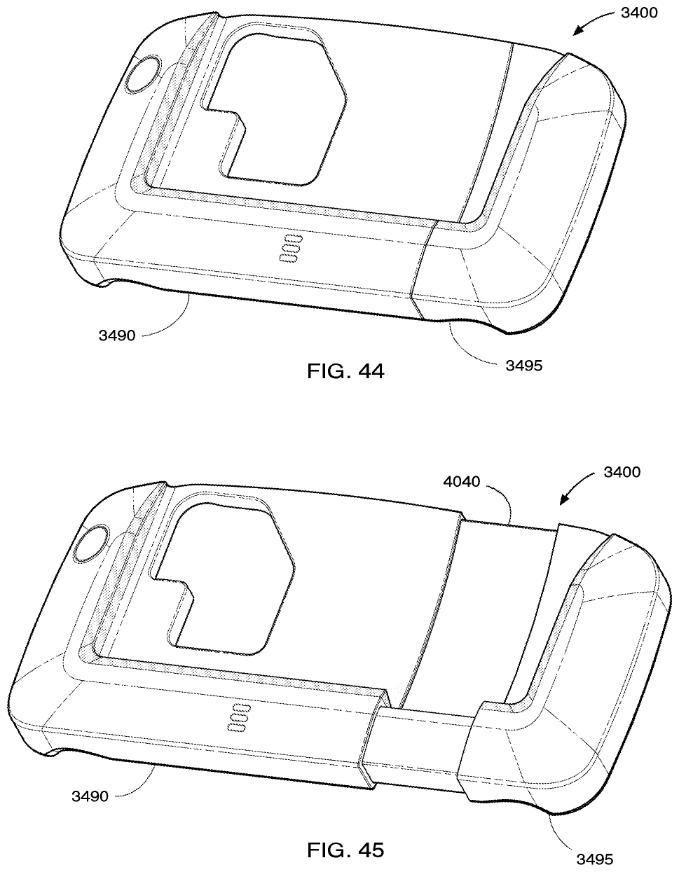

FIG. 44 shows a perspective view of image distance control device 3400 with the extendable portion (including second part 3495) in the closed position. FIG. 45 shows a perspective view of image distance control device 3400 with extendable portion (including second part 3495) in the extended position.

In another embodiment, the image distance control device gathers and stores information relating to the settings of the camera when a particular image is captured. The device may access these stored settings in connection with a subsequent image to ensure that the settings are consistent between two images. The device may display the settings to enable a user to adjust the settings of the camera for the subsequent image. In various embodiments, the device may store a checklist of default settings or information specific to the camera, such as information relating to the flash, iso, shutter speed, etc., and other parameters related to photographs or images.

Thus, in accordance with embodiment, an apparatus for measuring a distance between an imaging device and a wound is provided. The device includes a photographic device holder portion, a distance measurement device, and a light source.

In one embodiment, the photographic device holder portion includes a first region having a first surface at a first level, and a second region having a second surface at a second level different from the first level. The first region and second region are adapted to hold a photographic device, such as a cellular telephone, in place on the apparatus. The apparatus may include a main body section and an extendable section adapted to slide in and out relative to the body section in order to accommodate different sizes of cellular telephones.

In one embodiment, a wall is disposed at a boundary of the second region and between the first region and the second region. A layer of an elastomeric material is disposed between the wall and the first region.

In another embodiment, the apparatus includes a stem section, a middle arm section that is joined to the stem section and is perpendicular to the stem section, and a lower arm section that is joined to the stem section, is perpendicular to the stem section, and is parallel to the middle arm section.

In another embodiment, the apparatus includes a first region having a first surface at a first level, wherein a first portion of the first region is located on the stem section, a second portion of the first region is located on the middle arm section, and a third portion of the first region is located on the lower arm section. The apparatus also includes a second region having a second surface at a second level different from the first level, the second region having a fourth portion located on the stem section and a fifth portion located on the lower arm section. The apparatus also includes a third region having a third surface at a third level different from the first level, the third region being located on the middle arm section. The first region, second region, and third region are adapted to hold a photographic device, such as a cellular telephone, in place on the apparatus.

In another embodiment, the distance measurement device includes a laser measurement device.

In another embodiment, the light source includes one of an incandescent light source, a fluorescent light source, and a light emitting diode (LED) device.

In accordance with another embodiment, a method is provided. A photographic device, such as a cellular telephone, is secured in an image distance control device. The image distance control device is positioned near a wound that is to be imaged. A position of the image distance control device is adjusted until the image distance control device indicates that the distance from the image distance control device to the wound equals a predetermined distance. The photographic device (e.g., the cellular telephone) secured in the image distance control device is employed to generate an image of the wound.

In other embodiments, an image distance control device may have a shape, a design, and/or a configuration different from those described herein and different from those shown in the Drawings. Thus, in accordance with another embodiment, an apparatus for measuring a distance includes a distance measurement device, a light source, and a photographic device holder portion. The photographic device holder portion includes a first region having a first surface at a first level, and a second region having a second surface at a second level different from the first level. The first region and second region are adapted to hold a photographic device, such as a cellular telephone, in place on the apparatus.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. An apparatus for measuring a distance, the apparatus comprising:
a photographic device holder portion;
a distance measurement device; and
a light source.

2. The apparatus of claim 1, wherein the photographic device holder portion comprises:
a first region having a first surface at a first level; and
a second region having a second surface at a second level different from the first level;
wherein the first region and second region are adapted to hold a photographic device in place on the apparatus.

3. The apparatus of claim 2, wherein the photographic device holder portion is adapted to hold a cellular telephone.

4. The apparatus of claim 2, further comprising:
a wall disposed at a boundary of the second region and between the first region and the second region; and
a layer of an elastomeric material is disposed between the wall and the first region.

5. The apparatus of claim 2, wherein the distance measurement device comprises one of a laser measurement device and an infrared distance measurement device.

6. The apparatus of claim 2, wherein the light source includes one of an incandescent light source, a fluorescent light source, and a light emitting diode (LED) device.

7. The apparatus of claim 2, further comprising:
a first section; and
an extendable portion adapted to retractably extend relative to the first section, thereby increasing a surface area of the first region.

8. An apparatus for measuring a distance, the apparatus comprising:
a photographic device holder portion;
a distance measurement device; and
a light source;
wherein the apparatus includes:
a stem section;
a middle arm section that is joined to the stem section and is perpendicular to the stem section; and
a lower arm section that is joined to the stem section, is perpendicular to the stem section, and is parallel to the middle arm section.

9. The apparatus of claim 8, wherein the apparatus further includes:
a first region having a first surface at a first level, wherein a first portion of the first region is located on the stem section, a second portion of the first region is located on the middle arm section, and a third portion of the first region is located on the lower arm section;
a second region having a second surface at a second level different from the first level, the second region having a fourth portion located on the stem section and a fifth portion located on the lower arm section; and
a third region having a third surface at a third level different from the first level, the third region being located on the middle arm section;
wherein the first region, second region, and third region are adapted to hold a photographic device in place on the apparatus.

10. The apparatus of claim 8, wherein the distance measurement device comprises a laser measurement device.

11. The apparatus of claim 8, wherein the light source includes one of an incandescent light source, a fluorescent light source, and a light emitting diode (LED) device.

12. The apparatus of claim 8, wherein the photographic device holder portion is adapted to hold a cellular telephone.

13. A method comprising:
securing a photographic device in an image distance control device;
positioning the image distance control device near a wound that is to be imaged;
adjusting a position of the image distance control device until the image distance control device indicates that the distance from the image distance control device to a wound equals a predetermined distance; and
employing the photographic device secured in the image distance control device to generate an image of the wound.

14. The method of claim 13, wherein the image distance control device includes:
a photographic device holder portion;
a distance measurement device; and
a light source.

15. The method of claim 14, wherein the image distance control device further includes:
a stem section;
a middle arm section that is joined to the stem section and is perpendicular to the stem section; and
a lower arm section that is joined to the stem section, is perpendicular to the stem section, and is parallel to the middle arm section.

16. The method of claim 15, wherein the image distance control device further includes:
a first region having a first surface at a first level, wherein a first portion of the first region is located on the stem section, a second portion of the first region is located on the middle arm section, and a third portion of the first region is located on the lower arm section;
a second region having a second surface at a second level different from the first level, the second region having a fourth portion located on the stem section and a fifth portion located on the lower arm section; and
a third region having a third surface at a third level different from the first level, the third region being located on the middle arm section;
wherein the first region, second region, and third region are adapted to hold a photographic device in place on the apparatus.

17. The method of claim 13, wherein the distance measurement device comprises a laser measurement device.

18. The method of claim 17, wherein the light source includes one of an incandescent light source, a fluorescent light source, and a light emitting diode (LED) device.

19. The method of claim 13, wherein the photographic device comprises a cellular telephone.
